(19) United States
(12) Patent Application Publication
(10) Pub. No.: US 2025/0154200 A1
(43) Pub. Date: May 15, 2025

(54) CYCLIC PEPTIDE FOR CANCER IMMUNOTHERAPY

(71) Applicant: Kansas State University Research Foundation, Manhattan, KS (US)

(72) Inventors: Jeffrey COMER, Manhattan, KS (US); Ravindra THAKKAR, Manhattan, KS (US); Masaaki TAMURA, Manhattan, KS (US)

(21) Appl. No.: 18/722,010

(22) PCT Filed: Mar. 31, 2022
(86) PCT No.: PCT/US2022/022750

§ 371 Date: Jun. 20, 2024

Related U.S. Application Data
(60) Provisional application No. 63/291,522, filed on Dec. 20, 2021.

(51) Int. Cl. C07K 7/64; A61K 38/00; A61P 35/00; G01N 33/569

(52) U.S. Cl. CPC C07K 7/64; A61P 35/00; G01N 33/56972; A61K 38/00

(57) ABSTRACT

Cancer immunotherapies comprising synthetic oligopep-tide-based immune checkpoint inhibitors, methods of treating cancers using the same, and kits and compositions for treatment.

### CROSS-REFERENCE TO RELATED APPLICATIONS

The present application claims the priority benefit of U.S. Provisional Patent Application Ser. No. 63/291,522, filed Dec. 20, 2021, entitled CYCLIC PEPTIDE FOR CANCER IMMUNOTHERAPY, incorporated by reference in its entirety herein.

### FEDERALLY SPONSORED RESEARCH OR DEVELOPMENT

This invention was made with government support under DMR-1945589 awarded by the National Science Foundation. The government has certain rights in the invention.

### SEQUENCE LISTING

The following application contains a sequence listing submitted as an ASCII text file via EFS-Web in computer readable format (CRF) to serve as both the paper copy and CRF in compliance with 37 CFR 1.821. The ASCII text file is entitled “Sequence_Listing,” created on Mar. 30, 2022, as 9,472 bytes, and the content of the ASCII text file is incorporated by reference herein.

### BACKGROUND OF THE INVENTION

### Field of the Invention

The present invention relates to immune checkpoint inhibitor peptides for treatment of cancer.

### Description of Related Art

The cytotoxic T-lymphocyte-associated antigen 4 (CTLA-4) and programmed death 1 (PD-1) immune checkpoints are negative regulators of T-cell immune function. They prevent the immune system from attacking cells indiscriminately and play an important role in self-tolerance and deactivation of immune responses. The CTLA-4 and PD-1 immune checkpoint pathways downregulate T-cell activation to maintain peripheral tolerance.

In particular, CTLA-4 is a protein found on T cells, and when CTLA-4 is bound to another protein from the B7 family (CD80/CD86) on an antigen presenting cell (APC) or dendritic cell (DC), it helps keep T cells from killing other cells (i.e., the T cell remains inactivated). Conversely, T cell activation is initiated by the interaction between CD28 receptors on the T cells and B7 on the APC or DC. Thus, CTLA-4 shares the same B7 ligands as CD28, including B7-1 (CD80) and B7-2 (CD86) with negative effects on T cell activation. Moreover, the CTLA-4 receptor competes with CD28 for B7 binding and has a higher affinity for B7 than CD28. The B7 family consists of structurally related, cell-surface protein ligands, which bind to the CD28 family of receptors on lymphocytes and regulate immune responses via ‘costimulatory’ or ‘coinhibitory’ signals. The seven known members of the B7 family—B7.1 (CD80), B7.2 (CD86), inducible costimulator ligand (ICOS-L), programmed death ligand 1 (PD-L1), programmed death ligand 2 (PD-L2), B7-H3, and B7-H4—are all transmembrane or glycosylphosphatidylinositol (GPI)-linked proteins characterized by extracellular IgV and IgC domains related to the variable and constant domains of immunoglobulins. Binding of B7-1 (CD80) or B7-2 (CD86) molecules on APCs with CD28 molecules on the T cell leads to signaling within the T cell, thus stimulating the immune response. However, when the B7 protein is already bound by CTLA-4, it cannot then bind with CD28 or participate in this process.

Cancer cells can leverage these pathways to facilitate tumor growth and metastasis. Cancer cells often produce an abundance of CTLA-4 proteins. The association of CTLA-4 with proteins of the B7 family, such as CD80 and CD86, thus blocks their interaction with CD28 thereby blocking stimulation and activity of the T cells. In this manner, many types of cancer cells can evade destruction by the immune system.

Hence, blocking of CTLA-4 provides an opportunity for APC B7-2 to interact with CD28, to activate the T cells and stimulate the immune response. Understanding of these mechanisms has led to the development of new anticancer drugs, called immune checkpoint inhibitors, that are used to block CTLA-4 leading to activation of the immune system. Inhibitors of CTLA-4 can restore antitumor immune responses. Approved immunotherapies of this kind include ipilimumab (anti-CTLA-4), tremelimumab (anti-CTLA-4), pembrolizumab (anti-PD-1), and nivolumab (anti-PD-1).

Notably, there is still a lack of understanding of the underlying mechanisms, and these new therapies are not effective in some patients. Thus, there remains a new for new immune checkpoint inhibitors that may provide potential benefit for patients suffering from a variety of cancers, such as those associated with high CTLA-4 expression, including melanoma, mesothelioma, nasopharyngeal carcinoma, non-small cell lung cancer, kidney cancer, prostate cancer, and head and neck cancers.

### SUMMARY OF THE INVENTION

In cancer immunotherapy, a patient's immune system is modified to destroy the cancer cells. Cytotoxic T lymphocytes have an ability to identify tumor-specific antigens and kill the cancer cells. But the CTLA-4 protein expressed on the surface of the T-lymphocyte is one of the regulators of immune system that interacts with the B-7 family protein and suppresses the ability of the cytotoxic T lymphocytes to kill the cancer cells in many types of solid cancers. Therefore, blocking of the interaction between the B7 family proteins and the CTLA-4 protein restores the ability of cytotoxic T lymphocytes to fight against the cancer.

The present invention is broadly concerned with immune checkpoint inhibitor peptides (oligopeptides) and methods related to treatment of any cancer in which inhibition of CTLA-4 has been shown to improve treatment outcomes, including where inhibition of CTLA-4 has been shown effective in stimulating antitumor responses. Embodiments described herein include immune checkpoint inhibitor oligopeptides comprising (consisting essentially or even consisting of) the sequence:
R10: EIDTVLTPTGWVAKRYS (SEQ ID NO:1).

The immune checkpoint inhibitor peptides can be used for novel cancer immunotherapies. The immune checkpoint inhibitor peptides feature a unique head to tail cyclic structure. The immune checkpoint inhibitor peptides bind with the CTLA-4 protein and blocks its association with CD80 and CD86 proteins. That is, the immune checkpoint inhibitor peptides bind the CTLA-4 receptor site, where CD80 or CD86 normally bind and hence the formation of the CTLA-4: CD80 and CTLA-4: CD86 complexes can be blocked and an immune response can be triggered against cancer. Blocking of CTLA-4 also provides an opportunity for B7-2 to interact with CD28, stimulating the immune response. Unlike existing monoclonal antibody-based checkpoint inhibitors and other biologics-based therapies, the current invention represents the first chemically synthetic product for use as an immune checkpoint inhibitor.

Also described herein are therapeutic compositions comprising a plurality of immune checkpoint inhibitor oligopeptides according to the various embodiments described herein which are dispersed in a pharmaceutically-acceptable carrier.

The present disclosure also describes kits comprising a plurality of immune checkpoint inhibitor oligopeptides according to the various embodiments described herein in a unit dosage form in a container, and instructions for administering the oligopeptides to a subject in need thereof.

Also described herein are methods of treating cancer by administering a plurality of the oligopeptides to a subject in need thereof, and various other uses of the oligopeptide in diagnostic kits, or medicaments for treating cancer, inhibiting growth of cancer cells, or enhancing cancer treatment.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a cartoon depiction of a cyclic peptide according to an embodiment of the invention.

FIG. 2 is an image of a computer molecular dynamics simulation showing positioning of the cyclic peptide relative to the CTLA-4 receptor protein.

FIG. 3 is a graph comparing the MD simulation duration for different peptides and the B7-2/CD86 control for 2000 ns simulations that were terminated early if the ligand became unbound.

FIG. 4 is an annotated snapshot of a simulation showing the R10 peptide (green carbon atoms, Tyr16, Ala13, Arg15, and Lys14) bound to the CTLA-4 (gray carbon atoms for select residues Try104, Thr53, Glu48, Asp64, and gray secondary structure). Selected interactions between CTLA-4 and the peptide residues are shown as black dotted lines.

FIG. 5 is a graph showing the time-based binding kinetics for a bio-layer interferometry experiment for different molar concentrations of the peptide R10, with CD86 protein as a positive control, and PBS solution as a negative control performed. In cases, biotinylated recombinant human CTLA-4 Fc Avi-tagged protein had been loaded on the biosensor tip. The binding response was set zero at the beginning of the association, and the dissociation phase was initiated at 600 seconds.

FIG. 6 is a graph showing the average % of dead cells after different treatments, demonstrating that the checkpoint inhibitor peptide (R10; CTLA-4ip) enhanced cytotoxicity of antigen primed CD8+ T cells toward LLC cells. LLC cells were cocultured with antigen primed CD8+ T cells with 1:16 ratio. The cells were treated with the designed peptide R10 (10 μM) or anti-PD-L1 antibody (αPD-L1; 0.5 or 1.0 μg/ml) immediately after coculture. After 18 and 36 hrs of coculture, dead cells were identified by flow cytometry. Results are presented as mean±SD (n=2). a-c, P<0.05 between different characters.

FIG. 7 shows graphs of the data for number and volume of tumor nodules in mice administered various treatments. The checkpoint inhibitor peptide (R10; CTLA-4ip) attenuated the growth of LLC tumors in mouse lungs. LLC inoculated mice were intravenously injected with JAWSII cells stimulated by coculturing with irradiated LLC (JAWS-irrLLC) at 5 days after LLC inoculation. Mice were randomly divided into 3 groups and intraperitoneally treated with PBS (JAWS-irrLLC alone), CTLA-4 inhibitory peptide (R10, 10 mg/kg/day) and anti-PD-L1 antibody (αPD-L1, 10 mg/kg/day) as scheduled in Materials and Methods. The number of tumor nodules and its size were recorded at end of experiment. A-B: The number of tumor nodules in each mouse (A) and the average number in each group (B). C-D: The volume of tumor in each mouse (C) and the average volume in each group (D). B and D: Results are presented as mean±SD (n=5-6).

FIG. 8 shows graphs of characteristics of the cell populations following various treatments. Percent population of CD4+ FoxP3+ regulatory T cells and CD8+ IFNγ+ cytotoxic T cells, and the expression of immune check point molecules in mouse blood. (A) Average % population of CD4+ FoxP3+ regulatory T cells (Treg) and CD8+ IFNγ+ cytotoxic T cells (IFNγ). (B) Average mean fluorescence intensity (MFI) of IFNγ and PD-1 in CD8+ cytotoxic T cells. (C) Average MFI of CTLA-4 in CD4+ T cells (open bar) and Treg (filled bar). (D) Average MFI of CTLA-4 in CD8+ cytotoxic T cells (open bar) and CD8+ IFNγ+ activated cytotoxic T cells (filled bar).

FIG. 9A-D shows correlation diagrams between tumor growth, immune cell population and expression of immune checkpoint molecules in mice treated with PBS (JAWS-irrLLC alone), CTLA-4 inhibitory peptide (R10, 10 mg/kg/day) and anti-PD-L1 antibody (αPD-L1, 10 mg/kg/day), including comparison of tumor volume to (A) CD4+ FoxP3+ regulatory T cell (Treg) and CD8+ IFNγ+ cytotoxic T cell (IFNγ) populations; (B) MFI of IFNγ and PD-1 in CD8+ T cells; (C) MFI of CTLA-4 in CD4+ T cell; and (D) MFI of CTLA-4 in Treg.

FIG. 9E-F shows correlation diagrams between tumor growth, immune cell population and expression of immune checkpoint molecules in mice treated with PBS (JAWS-irrLLC alone), CTLA-4 inhibitory peptide (R10, 10 mg/kg/day) and anti-PD-L1 antibody (αPD-L1, 10 mg/kg/day), including comparison of tumor volume to (E) MFI of CTLA-4 in CD8+ cytotoxic T cells, and (F) MFI of CTLA-4 in CD8+ IFNγ+ activated T cells.

FIG. 10 shows graphs of surface expression of CTLA-4 in primary cultured murine NK cells at 48 h and 72 h following different treatments.

FIG. 11 shows graphs of intracellular expression of CTLA-4 in primary cultured murine NK cells at 48 h and 72 h following different treatments.

FIG. 12 shows graphs of cytotoxicity of H1N1 infected CT26 cells following different treatments at 48 h and 72 h.

FIG. 13A shows graphs of the peptide stability over time in cell culture media.

FIG. 13B shows graphs of the peptide stability over time in cell lysate.

### DETAILED DESCRIPTION

The present invention is concerned with therapeutic peptides that can be used to enhance immune responses against cancer cells, including enhancing a local immune response in cancer tissue, or enhance effectiveness of a cancer treatment by administration of the peptides as immune checkpoint inhibitors. The peptide (referred to as R10) blocks the interaction of CTLA-4 with B7 proteins, leaving the B7 protein free to interact with CD28 on T cells, thus triggering the immune response against cancer cells in the patient. The peptide comprises the sequence:
R10: EIDTVLTPTGWVAKRYS (SEQ ID NO:1)

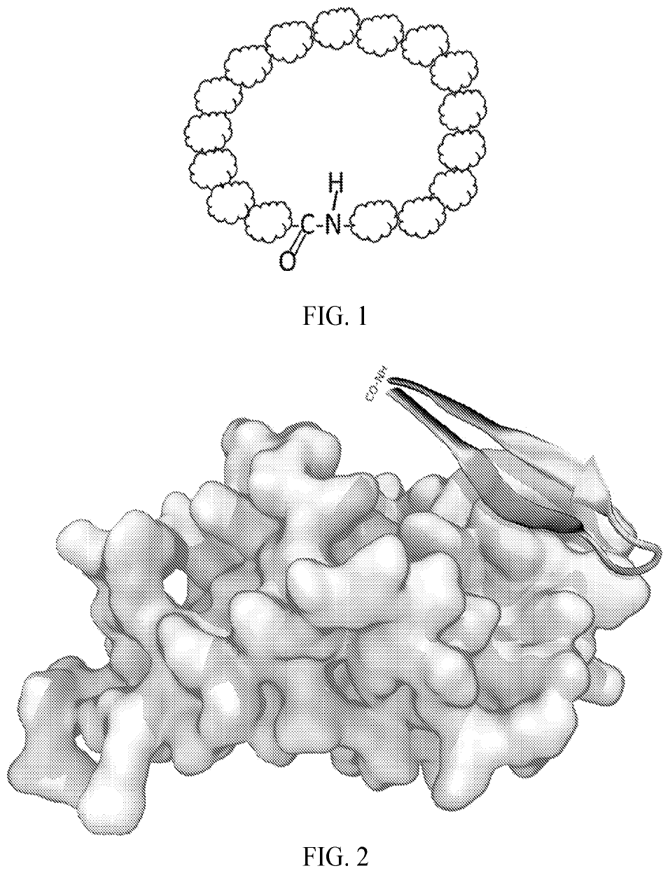

Preferably, the peptide is a cyclic peptide (e.g., the N- and C-termini are linked by, for example, a peptide bond, or the presence of intermolecular cyclizations). Preferably, the cyclic peptide is one in which the amino acid residues are arranged in a circular sequence, with the N-terminal head connected to the C-terminal tail via a peptide bond (e.g., amide bond, —C(═O)—NH—), as illustrated in FIG. 1. It will be appreciated because the resulting cyclic peptide is head-to-tail cyclic, there is subsequently no “N-terminus” or “C-terminus” in the cyclized peptide, but rather the recited amino acid residues are merely connected in order via respective peptide bonds in a circular, ovoid, or annular ring. Moreover, it should be noted that peptide synthesis can be started from any of the desired residues in the disclosed sequence, and should not be construed as being limited to synthesizing, for example, beginning with the Glutamic Acid (E) residue shown as the “first” or N-terminal residue above. Thus, in terms of a cyclic peptide, cyc(EIDTVLTPTGWVAKRYS) (SEQ ID NO:2) is the same peptide as cyc(IDTVLTPTGWVAKRYSE) (SEQ ID NO:3) or cyc(DTVLTPTGWVAKRYSEI) (SEQ ID NO: 4) or any other cyclic permutation, for a total of 17 equivalent representations depending upon how the linear arrangement of the amino acid residues are depicted, see SEQ ID NOs: 2-17.

Synthetic peptides can also be cyclized by one or more disulfide bridges in lieu of peptide bonds. Furthermore, crosslinks of various chemistries (referred to as “staples”) can be used to cyclize peptides in ways never found in nature. It will be appreciated that other approaches can be used to synthesize cyclic peptides, including side chain-to-side chain bonds (e.g., usually Lys, Asp, or Glu), head-to-side chain bonds, and side chain-to-tail bonds. Other suitable peptide linkers may also be used. The peptides can be prepared by various synthetic methods of peptide synthesis, such as via condensation of one or more amino acid residues, in accordance with conventional peptide synthesis methods. Synthesis can also be carried out using standard solid-phase methodologies, such as may be performed on a commercial automatic synthesizer. Other methods of synthesizing peptides, either by solid-phase methodologies or in liquid phase, are well known to those skilled in the art and are contemplated herein, such as by Boc-mediated solid-phase peptide synthesis or Fmoc-based chemistries.

The peptide has an affinity and specificity for binding CTLA-4. More preferably, the peptide has an affinity and binds to at least the conserved motif MYPPPY (SEQ ID NO:18) of the CTLA-4 receptor. In one or more embodiment, residues Pro8, Ala13, Arg15, and Tyr16 of R10: EIDTVLTPTGWVAKRYS (SEQ ID NO:1) participate strongly in binding.

In one or more embodiments, analogs or derivatives of SEQ ID NO:1 are contemplated herein. Analogs or derivatives may comprise conserved amino acid substitutions (preferably 2 or less) and or side chain functionalization. In one or more embodiments, a conserved amino acid substitution may be made at any one or more of residues Glu1, Ile2, or Asp3. In one or more embodiments, Glu1 may be substituted for a Cys residue. In one or more embodiments, the peptide may comprise a functional moiety or label may be attached to a side chain of any one or more of residues Glu1, Ile2, or Asp3. In one or more embodiments, Glu1 (or conserved substitution Cys1) may comprise a detectable moiety such as a fluorescent dye or peptide. Fluorescently labeled peptides can be used for cell culture (microscopic observation), flow cytometry, and histological observation (e.g., with confocal microscope). Peptide modifications are known in the art, including N-terminal (e.g., biotin, 5-FAM, Abz, Boc, CBZ, Fmoc, and the like) or C-terminal modifications (e.g., AFC, AMC, Amidation, Esterification, and the like), stable isotope labels (e.g., Ile(13C6,15N), Lys(13C6,15N2), Val(13C5,15N), and the like), fluorescent moieties (5-FAM, Abz, DABCYL, Fluorescein isothiocyanate (FITC), MCA, and the like).

In one or more embodiments, the peptides can be modified or functionalized for targeting cancer tissues, such as by attaching a targeting moiety or ligand having affinity for cancer cells, such as RGD peptides which preferentially accumulate near cancer tissue. It will also be appreciated that the affinity of the peptides for CTLA-4 receptor provides the peptides with a cancer targeting ability. Thus, in some embodiments, the cyclic peptides may themselves be considered tumor-targeting moieties that could be attached to other active agents.

The peptides have also shown an affinity for T cells (e.g., via CD28) and can be used for kits and assays to detect the presence and/or quantity of Cytotoxic T-Lymphocytes. Thus, the peptides can be used as a tracking tool for both T cells (including both CD4+ and CD 8+ T cells) and NK cells. It will be appreciated that such assays have the advantage of the small size of the peptides (approximately 2,000 Da). In contrast, since the molecular sizes of antibodies (e.g., IgG, ˜150 Kda) and most fluorescent dyes that are commonly used for this purpose are much larger, these common marking molecules may change the cell's behaviors, unlike the peptides. Moreover, the peptides can be conjugated with fluorescent dyes by side chain modifications such that cells with which the peptides interact can be detected and quantified based upon the strength of the detectable fluorescent signal (which can be detected, e.g., using fluorescent microscopy or flow cytometry). As an exemplar assay, fluorescent dye conjugated peptides are prepared using commercially available service. In order to detect CTLA-4 expressing CTL in mouse spleen (or blood), splenocytes (or PBMC (peripheral blood mononuclear cells)) are reacted with fluorescent dye conjugated peptide and anti-CD8b antibody. CTLA-4+ and CD8+ CTL is detected by fluorescent microscope or flow cytometry based upon the strength of the fluorescent signal. Likewise, cells that interact with the peptides can be identified or quantified by synthesizing the peptides using radiolabeled (tritium (3H) or carbon 14 (14C); both 3 and 14 next to H or C must be superscript) or stable isotope-labeled amino acids, followed by quantification of the detected radioactivity by a liquid scintillation counter or mass spectrometry for stable isotopes (amount of radioactivity correlates the quantity of cells).

For therapeutic, diagnostic, or theranostic uses, the peptides are typically administered as part of a composition comprising a plurality of peptides dispersed in a pharmaceutically-acceptable carrier. The term carrier is used herein to refer to diluents, excipients, vehicles, and the like, in which the peptide may be dispersed for administration. Suitable carriers will be pharmaceutically acceptable. As used herein, the term “pharmaceutically acceptable” means not biologically or otherwise undesirable, in that it can be administered to a subject without excessive toxicity, irritation, or allergic response, and does not cause unacceptable biological effects or interact in a deleterious manner with any of the other components of the composition in which it is contained. A pharmaceutically-acceptable carrier would be selected to minimize any degradation of the peptide or other agents and to minimize any adverse side effects in the subject. Pharmaceutically-acceptable ingredients include those acceptable for veterinary use as well as human pharmaceutical use and will depend on the route of administration. For example, compositions suitable for administration via injection are typically solutions in sterile isotonic aqueous buffer. Exemplary carriers include aqueous solutions such as normal (n.) saline (˜0.9% NaCl), phosphate buffered saline (PBS), sterile water/distilled autoclaved water (DAW), various oil-in-water or water-in-oil emulsions, as well as dimethyl sulfoxide (DMSO), other acceptable vehicles, and the like.

The composition can comprise a therapeutically effective amount of the peptide dispersed in the carrier. As used herein, a “therapeutically effective” amount refers to the amount that will elicit the biological or medical response of a tissue, system, or subject that is being sought by a researcher or clinician, and in particular elicit some desired therapeutic effect as against the cancer cells by blocking the interaction of CTLA-4 with B7 proteins, leaving them free to interact with and activate the immune cells. Thus, the peptides are preferably provided in an amount sufficient to block a suitable quantity of CTLA-4 to facilitate activation of an effective amount of immune cells against the cancer cells. One of skill in the art recognizes that an amount may be considered therapeutically “effective” even if the condition is not totally eradicated or prevented, but it or its symptoms and/or effects are improved or alleviated partially in the subject, such as reduction in number/volume of cancer cells or tissue/tumor nodules and/or reduction in rate of growth of the cancer cells, or tissue/nodules even if the cancer is not totally eradicated. That is, the immune checkpoint inhibitor peptides can be useful in enhancing the immune response against the cancer and reducing or stalling the cancer, such that adjunct therapies such as chemotherapy or other immunotherapy, and the like can have a greater impact on thereafter eradicating the cancer cells. Thus, it is contemplated that the immune checkpoint inhibitor peptides may be used as part of a multi-faceted cancer treatment plan. In some embodiments, the composition will comprise from about 5% to about 95% by weight of the peptides described herein, and preferably from about 30% to about 90% by weight of the peptides, based upon the total weight of the composition taken as 100% by weight. Encapsulation techniques can also be used to facilitate delivery of the peptides.

Other ingredients may be included in the composition, such as adjuvants, other active agents (e.g., other checkpoint inhibitors, immunotherapies, chemotherapies), preservatives, buffering agents, salts, other pharmaceutically-acceptable ingredients. The term “adjuvant” is used herein to refer to substances that have immunopotentiating effects and are added to or co-formulated in a therapeutic composition in order to enhance, elicit, and/or modulate the innate, humoral, and/or cell-mediated immune response against the active ingredients. Other active agents that could be included in the composition include any immunogenic active components (e.g., monoclonal antibodies, tumor antigens, etc.), such that it provokes a general increase in immune response in the patient and/or a targeted immune response against the cancer cells.

In use, a therapeutically-effective amount of peptide is administered to a subject. In some embodiments, a composition comprising a therapeutically-effective amount of peptide is administered to a subject. The disclosed embodiments are suitable for various routes of administration, depending upon the particular carrier and other ingredients used. For example, the peptides can be injected intramuscularly, intraperitoneally, subcutaneously, intradermally, or intravenously. They can also be administered via mucosa such as intranasally or orally. The compounds or compositions can also be administered through the skin via a transdermal patch, or topically applied to dermal and epidermal-based cancers (e.g., melanoma). In one or more embodiments, the peptide is formulated for intratumoral administration, wherein the peptide is locally injected in or near the site of cancer cells or a tumor. Intratumoral administration of the peptide results in changes in the tumor microenvironment, including binding CTLA-4 and subsequent activation of the immune system (through interaction of CD28 and B7). As noted, the peptides can be administered alone or co-administered with other immunotherapies and/or chemotherapies. In one or more embodiments, coadministration means simultaneous administration of two or more active agents, either in the same composition or at the same time but in respective compositions (e.g., separate injections, or into the same IV drip line, etc.). Coadministration may also refer to sequential administration of the active agents, separated by minutes or hours, but typically within the same day (24 hour period). In one or more embodiments, the checkpoint inhibitor peptides may be administered as part of a multi-faceted cancer treatment program for a subject. In one or more embodiments, the checkpoint inhibitor peptides may be used as the primary cancer treatment. In one or more embodiments, the checkpoint inhibitor peptides may be used as an adjunctive treatment. Adjunctive treatments are typically those given after the primary treatment to lower the risk that the cancer will come back. Additional adjunctive treatments that may be administered alongside of the checkpoint inhibitor peptides may include chemotherapy, radiation therapy, hormone therapy, or immunotherapy. Those skilled in the art can develop the appropriate treatment plan based upon the particular cancer involved, the stage of the cancer, prognosis, and age of the patient.

Upon administration the checkpoint inhibitor peptides bind to and block CTLA-4 and prevent its binding to the B7 family of proteins, leaving them free to interact with CD28 and in turn activate the immune system enabling the immune system to recognize tumor cells and allowing a sustained immunotherapy response (preferably lasting at least 24 hours after administration). Advantageously, the checkpoint inhibitor peptides have been shown to be incredibly stable and resistant to enzymatic degradation. Moreover, unlike linear peptides, which are completely degraded by 72 hours, the checkpoint inhibitor cyclic peptides are not only taken up by cells, but remain intact in the cells to provide immune checkpoint inhibition for at least 72 hours without significant degradation. Embodiments described herein thus include methods of inducing an immune response to cancer cells. Also described herein are methods of activating an immune cell at a cancer site comprising cancer cells.

In some embodiments, the checkpoint inhibitor peptides (or compositions) can be provided in unit dosage form in a suitable container. The term “unit dosage form” refers to a physically discrete unit suitable as a unitary dosage for human or animal use. Each unit dosage form may contain a predetermined amount of the checkpoint inhibitor peptides (and/or other active agents) in the carrier calculated to produce a desired effect. In other embodiments, the checkpoint inhibitor peptides can be provided separate from the carrier (e.g., in its own vial, ampule, sachet, or other suitable container) for on-site mixing before administration to a subject.

A kit comprising the checkpoint inhibitor peptides is also disclosed herein. The kit further comprises instructions for administering the checkpoint inhibitor peptides to a subject. The checkpoint inhibitor peptides can be provided as part of a dosage unit, already dispersed in a pharmaceutically-acceptable carrier, or provided separately from the carrier. The kit can further comprise instructions for preparing the checkpoint inhibitor peptides for administration to a subject, including for example, instructions for dispersing the checkpoint inhibitor peptides in a suitable carrier.

It will be appreciated that therapeutic and prophylactic methods described herein are applicable to humans as well as for veterinary use for any suitable animal, including, without limitation, dogs, cats, and other companion animals, as well as, rodents, primates, horses, cattle, pigs, etc. The methods can be also applied for clinical research and/or study.

Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination. The term “inhibitor” generally refers to a substance that can bind to a receptor, but does not produce a biological response upon binding. The inhibitor can block, inhibit, or attenuate the response mediated by an agonist and may compete with agonist for binding to a receptor. Such inhibitory activity may be reversible or irreversible. The term “enhance” means to add, increase, improve and/or intensify. In the case of the checkpoint inhibitor peptides, they enable enhancement of the immune response against the cancer cells in the patient as compared to a baseline immune response before or without administration of the peptides.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods in accordance with the invention. It is to be understood, however, that these examples are provided by way of illustration and nothing therein should be taken as a limitation upon the overall scope of the invention.

### EXAMPLE 1

### Peptide Design and Computational Modeling

Described herein is a cyclic peptide (R10) that binds with the CTLA-4 protein and blocks its association with B7 family proteins. The peptide was designed and tested via computational modeling. A fragment of the B7-2 protein (amino acid residues 85-101) was selected as a template for initial design of a peptide that will bind to CTLA-4 receptor with high affinity and specificity. Several sequences were developed. Computational models were used to predict protein-peptide docking based upon probable orientation of the peptide molecule on the protein surface. A docking score was calculated for the designed sequences based on an effective potential energy function. Peptides having a favorable conformation on the receptor protein surface were selected for further analysis. Based upon initial modeling, the designed peptide (R10) has a predicted 270 nanomolar binding affinity of with CTLA-4 protein. Additional modeling was used including RMSD (Root Mean Square Deviation) to analyze atomic positions between two possible conformations of the peptides. Peptides having values similar to the template and with lower energy score were selected for further analysis. Side-chain and rotamer optimization used different isomers of the same residue or change the residue peptide to make stronger interaction with the receptor protein. Rotamer and side-chain optimization of template peptide leads to closer contact with the receptor protein. See exemplary values in table below.

TABLE
A comparison of amino acid sequence and energy
score for complex between CTLA-4 and the peptides.

| | Energy | SEQ |
|---|---|---|
| Peptide Sequence | Score | ID NO: |
| Template CIIHHKKPTGMIRIHQM | 186.50 | 19 |
| Designed (R10) EIDTVLTPTGWVAKRYS | 157.61 | 1 |

Next, to improve conformational and proteolytic stability and other characteristics, we simulated connecting the C-terminus and N-terminus of the designed peptides with a peptide bond (—C(═O)—NH—), as illustrated in FIG. 2. These circular peptides were then used for molecular dynamics simulations (using the software NAMD) to analyze the motion of a biomolecular system under the influence of the force generated by atomic interactions so that we could view the molecular interaction at the atomic level.

TABLE
MD simulation and binding free energy of cyclic peptides

| Protein Ligand | Binding Free Energy (kcal/mol) |
|---|---|
| CD-86 | −24.3 |
| R10 | −12.6 |
| 72_195_T (R7) | −32.8 |
| 72_33_T | −24.5 |
| 72_123 | −27.2 |

MM-GBSA Method for estimating binding free energy.

The binding affinities of the peptides for the target receptor protein were predicted using binding free energy calculation. The simulations were set to stop when the ligand molecule (B7-2/designed peptide) starts unbinding from the receptor protein (CTLA-4).

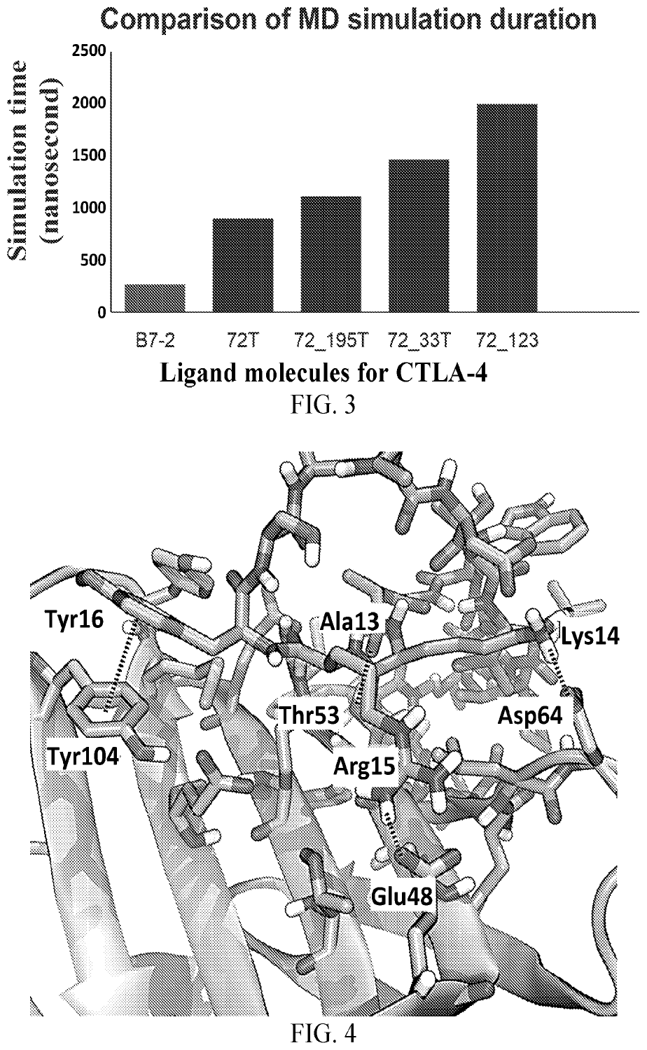

FIG. 3 shows a bar plot of the simulation time (nanoseconds) with different ligand molecules for receptor protein CTLA-4. B7-2 protein (a natural binding partner of CTLA-4) is shown in comparison to the designed peptides. The simulations, with a maximum time of 2000 ns, were stopped if the ligand dissociated from CTLA-4. In the MD simulations the candidate peptides were bound with CTLA-4 for more than a microsecond until the structure of peptide deviates by 25 Å from its native conformation. This shows the structural stability of the candidate peptides during its interaction with CTLA-4. We performed the free energy calculations by MM-GBSA method to shortlist the optimized peptide and performed a rigorous free energy calculation by geometrical route using BFEE plugin of VMD. We shortlisted 6 optimized peptides which has MMGBS score more than 20 kcal/mol. Upon vigorous calculation, only two peptides showed a binding affinity of −10.21±2.41 and −12.58±3.76 kcal/mol respectively, which is sufficient to block the MYPPPY (SEQ ID NO:18) motif on the surface of the CTLA-4 receptor for B7 family proteins. The beta shaped structure of the cyclic peptide was maintained during the docking and sequence optimization steps, but during the MD simulations the B-structure for most of the peptide was lost.

TABLE
Characteristics of designed peptides for the
CTLA-4 receptor protein.

| | Optimization | | Duration | Free | Free |
|---|---|---|---|---|---|
| | Score | | of MD | Energy by | Energy by |
| | Pre | Post | Simulation | MMGBSA | BFEE |
| Sequence | design | design | (ns) | (kcal/mol) | (kcal/mol) |
| Cyc(ECRYEPRPEGNILVSYS) | 186.49 | 170.67 | 660.2 | −22.67 ± | +2.61 ± |
| SEQ ID NO: 20 | | | | 0.18 | 1.42 |
| Cyc(SIVTKLTPTGWVAASYS) | 185.57 | 175.15 | 899.4 | −26.40 ± | +3.51 ± |
| SEQ ID NO: 21 | | | | 0.14 | 13.24 |
| Cyc(KVEFKRTPSGTITVSME) | 176.95 | 165.86 | 12.8 | −10.43 ± | N/A |
| SEQ ID NO: 22 | | | | 0.64 | |
| Cyc(KVVYEPKPEGNIVVEYE) | 211.67 | 194.39 | 48.4 | −12.42 ± | N/A |
| SEQ ID NO: 23 | | | | 0.34 | |
| Cyc(SAKFEPRPEGNIVVSYG) | 202.6 | 200.6 | 134 | −22.67 ± | N/A |
| SEQ ID NO: 24 | | | | 0.24 | |
| Cyc(EARYQPRPDGNVLVSYG) | 204.99 | 206.02 | 254.6 | −14.20 ± | N/A |
| SEQ ID NO: 25 | | | | 0.19 | |
| Cyc(SAKWNPKPEGAELIEEG) | 226.5 | 222.82 | 16 | −7.69 ± | N/A |
| SEQ ID NO: 26 | | | | 0.67 | |
| Cyc(SAEFIPTPDGNLLKSSG) | 216.4 | 214.02 | 13.8 | −8.79 ± | N/A |
| SEQ ID NO: 27 | | | | 0.72 | |
| Cyc(SIVVVLTPTGWVAASYS) | 155.77 | 157.63 | 278 | −26.94 ± | N/A |
| SEQ ID NO: 28 | | | | 0.29 | |
| Cyc(EIITKLTPTGWVAASYS) | 156.02 | 157.61 | 86 | −19.29 ± | N/A |
| SEQ ID NO: 29 | | | | 0.35 | |
| Cyc(SIEMELTPTGWVNKSSS) | 159.12 | 157.96 | 44.2 | −11.30 ± | N/A |
| SEQ ID NO: 30 | | | | 0.33 | |
| Cyc(SIITVLTPTGWVAAEFS) | 154.32 | 155.7 | 1110.6 | −32.76 ± | −10.21 ± |
| SEQ ID NO: 31 | | | | 0.14 | 2.41 |
| Cyc(DIITILTPTGYVAAAYS) | 152.66 | 154.02 | 395.6 | −19.01 ± | N/A |
| SEQ ID NO: 32 | | | | 0.18 | |
| Cyc(SIITVLTPTGWVAAYYS) | 153.1 | 155.36 | 1463.2 | −23.31 ± | +5.11 ± |
| SEQ ID NO: 33 | | | | 0.09 | 21.31 |
| Cyc(SIQCVLTPTGWVAARYS) | 153.69 | 155.15 | 42.4 | −23.59 ± | N/A |
| SEQ ID NO: 34 | | | | 0.71 | |
| Cyc(EIDTVLTPTGWVAKRYS) | 152 | 153.85 | 2000 | −22.30 ± | −12.58 ± |
| SEQ ID NO: 1 | | | | 0.11 | 3.76 |
| Cyc(SIRMELTPTGWVAAEYE) | 165.81 | 165.25 | 119.4 | −18.47 ± | N/A |
| SEQ ID NO: 35 | | | | 0.37 | |

MMGBSA-Molecular Mechanics Generalized Born Surface Area
BFEE-Binding Free Energy Estimator

FIG. 4 shows A snapshot from the MD simulation of CTLA-4 (grey) and one of the designed 5 peptides R10 (green carbon atoms, Tyr16, Ala13, Arg15, and Lys14), depicting the atomic interactions between the protein and peptide. The peptide binds near the conserved MYPPPY (SEQ ID NO:18) (residues) domain where the B7 family of proteins binds.

TABLE
List of the residues participating in H-bond, salt bridge, hydrophobic
and π-π interactions for peptide R10.

| | Peptide | |
|---|---|---|
| CTLA-4 | R10 | Interaction |
| Thr53 (OH) | Ala13 (═O) | H-bond |
| Arg35 (NH) | Arg15 (═O) | H-bond |
| Glu48 (O⁻) | Arg15 (NH) | salt bridge |
| Glu59 | Pro8 | hydrophobic |
| Tyr104 | Tyr16 | π-π interaction |

### EXAMPLE 2

### Peptide Synthesis—Binding Assay by Bio-Layer Interferometry

Based upon the computer simulations, we selected peptide R10 as the lead candidate. The cyclic peptide R10 was synthesized using a commercial synthesizer. Bio-layer interferometry-based binding assay was performed to determine the binding affinity of designed peptide R10 for CTLA-4 protein. For the experiments, Avi-tagged CTLA-4 and CD86 (B7-2) were also obtained from commercial sources. We measured the binding affinity by using Bio-Layer Interferometry based FortéBio's BLItz instrument, where PBS (phosphate buffer saline) without designed cyclic peptide (R10) was used to set up baseline and CD86 was used as positive control. Binding affinity was evaluated at room temperature. HIS1K BLItz biosensor tips (FortéBio; Freemont, CA, USA) were used to immobilize the CTLA-4 protein and all the tips were hydrated for 15-30 minutes in PBS buffer before each experiment. A constant signal at the washing (after loading the CTLA-4 on biosensor tips) indicated an immobilization of the CTLA-4 protein on the HIS1K biosensor tip. The PBS buffer without the designed cyclic peptide or any protein was used to record the baseline. 400 nM CD86 protein, a natural binding partner of CTLA-4 was used as positive control and different molar concentration (150,175 and 200 μM) of the designed peptide R10 as test analyte. The values of association and dissociation constant were obtained using the BLItz Pro 1.2 software.

The results obtained by in-built software shows cyclic peptide (R10) has 36±500 micromolar affinity for the CTLA-4 protein in vitro, as shown in the table below.

| Sample | Conc | KD | ka | ka | kd | kd | | Rmax | R |
|---|---|---|---|---|---|---|---|---|---|
| ID | (nM) | (M) | (1/Ms) | Error | (1/s) | Error | Rmax | Error | equilib. |
| PBS | 0 | — | — | — | — | — | — | — | — |
| R10- | 200000 | 3.061e−5 | 1.641e2 | 2.637el | 5.024e−3 | 5.938e−4 | 0.2097 | 0.02625 | 0.1819 |
| 400 ug | | | | | | | | | |
| R10- | 150000 | 3.061e−5 | 1.641e2 | 2.637e1 | 5.024e−3 | 5.938e−4 | 0.05944 | 0.005425 | 0.04936 |
| 300 ug | | | | | | | | | |
| R10- | 100000 | 3.061e−5 | 1.641e2 | 2.637el | 5.024e−3 | 5.938e−4 | 0.08469 | 0.01136 | 0.06484 |
| 200 ug | | | | | | | | | |
| R10- | 50000 | 3.061e−5 | 1.641e2 | 2.637el | 5.024e−3 | 5.938e−4 | 1.199e−31 | 0.002339 | 7.435e−31 |
| 100 ug | | | | | | | | | |
| R10- | 175000 | 3.061e−5 | 1.641e2 | 2.637e1 | 5.024e−3 | 5.938e−4 | 0.1059 | 0.009764 | 0.09011 |
| 350 ug | | | | | | | | | |
| CD86 | 0.3846 | 6.142e−8 | 1.289e5 | 5.189e3 | 7.917e−3 | 2.5e−4 | 0.3779 | 0.01203 | 0.3259 |

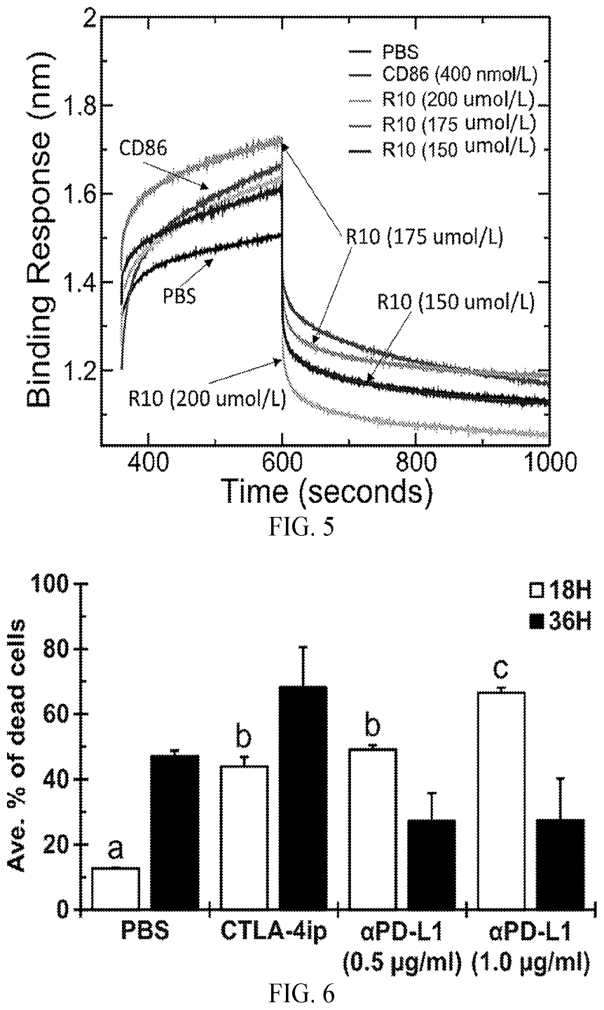

We confirmed that the biotinylated recombinant human CTLA-4 Fc Avi-tagged protein non-covalently was immobilized on the HIS1K biosensor tip. It was visible that the CTLA4 protein was immobilized on the HISK-1 biosensor tips whereas we did not get any binding response while applying PBS at the loading step (data not shown). Immobilization of CTLA4 on HIS-K1 biosensor tip was further confirmed when CD-86 was used as an analyte at the association step (data not shown). Our binding kinetics results showed 60±2.5 nM of dissociation constant (KD) for the h-CTLA4 to CD-86 interaction which is corroborate with the previous reports.⁶⁰ Thus, HIS1K biosensor tip and available CTLA4 protein could be used to evaluate the binding affinity of the designed peptide R10. The binding kinetics results for the designed peptide R10 are shown in FIG. 5. The (KD) value for the designed peptide R10 was found 30±5.9 μM, which is close to the predicted by BFEE calculation by MD simulations.

### EXAMPLE 3

### Peptide Synthesis—In Vitro and In Vivo Testing

We have confirmed the biological activity of newly designed cyclic peptide (CTLA-4inhibitory peptide, hereafter abbreviated as “CTLA-4ip”) by in vivo and in vitro studies.

Briefly, for in vitro studies, mouse Lewis Lung Carcinoma (LLC) cells were co-cultured with the LLC cell-antigen primed T cells in the presence of 10 μM CTLA-4ip, 0.5 μg/ml, 1.0 μg/ml anti-PD-L-1 antibody (αPD-L1, a proven therapeutic antibody was used as a positive control), or PBS (negative control). After 18- and 36-hours of incubation, the antigen primed T cell-induced cell death in cancer cells was identified by flow cytometry.

These in vitro study results showed that the effect of CTLA-4ip peptide was as strong as that by anti-PD-L1 antibody (0.5 μg/ml) at 18 hours after the incubation. Unexpectedly, the cancer cell killing effect by anti-PD-L1 antibody treatment declined to the level of the PBS control group, while the T cell-induced cancer cell killing effect of the CTLA-4ip was still strong at 36 hours later. This suggest that the therapeutic efficacy by this CTLA-4ip may last longer than the proven anti-PD-L1 antibody.

For in vivo studies, the effect of CTLA-4ip peptide on the tumor growth in the mouse lung was evaluated using an orthotropic LLC cell allograft model. After 5 days of LLC cell inoculation, all groups of mice were pretreated with JAWS II murine dendritic cells primed with irradiated LLC cells. Two days later mice were treated with either CTLA-4ip peptide (10 mg/kg/day, IP, every other day, 4 times), anti-PDL-1 antibody (10 mg/kg/day, IP, every two day, 3 times, as positive control), or PBS (negative control). Twenty-three days after the LLC cell inoculation, mice were sacrificed and tumor burdens were evaluated by microscopic and macroscopic observation.

The results indicated that the number and size of tumor nodules in the lungs were markedly smaller in CTLA-4ip peptide treated mouse group as compared to the PBS treated control. CTLA-4ip peptide treatment-induced reductions in both tumor nodule numbers and tumor volume were identical to those in the anti-PDL-1 antibody-treated group.

These mouse studies along with the in vitro cell culture study strongly suggest that this newly designed CTLA-4ip possesses therapeutic ability against lung cancer. Since the cancer growth in the animal body including human body is critically controlled by host anti-cancer immunity and the major players of the anti-cancer immunity are the cytotoxic T cells and NK cells, both of which express CTLA-4 as a negative regulator, this CTLA-4ip-based cancer therapy should be applicable to wide range of cancer types including both solid and blood cancers as well as various soft tissue- and osteo-sarcomas.

### Evaluation of the Effect of Designed Peptide R10 on T Cell-Induced Death of Murine Lung Carcinoma Cells in Co-Culture With Antigen-Primed T Cells

a. Generation of LLC cell antigen primed CD8+ T cells in vivo: Mice were subcutaneously (SQ) injected with LLC lysate (0.5×106 cells/mouse in 200 μl) at Day 0. X-ray (100 Gy) irradiated LLC cells were cocultured with JAWSII immature dendritic cells at a 1:1 ratio for 48 hrs with an additional treatment of LPS (1 μg/ml). The cells, named as JAWS-irrLLC, were collected and intravenously injected into mice via tail vein (0.5×104 cells/mouse in 200 μl) at Day 7. At Day 21, the splenocytes were harvested and CD8+ T cells were labeled using MojoSort™ Mouse CD8 T Cell Isolation Kit (Biolegend, San Diego, CA) and isolated using MACS® Column (Miltenyi Biotec, Bergisch Gladbach, Germany) according to the manufacturer's instructions. The purified CD8+ T cells were used as LLC cell antigen-primed CD8+ T cells (AP-CD8+ T cells).
b. Evaluation of T cell-induced death of lung carcinoma cells in co-culture: Permanently GFP-expressing LLC cells produced by GFP-lentivirus vector transduction (GFP-LLC cells) were seeded into 12-well plate (1×104 cells/well) and treated with murine interferon gamma (mIFNγ) at 25 ng/ml for 48 hrs. The cells were treated with 10 μM peptide R10 and 0.5 or 1.0 μg/ml mouse anti-PD-L1 antibody (αPD-L1) 30 min before coculture and AP-CD8+ T cells were added into each well at 1:16 ratio (LLC cells: AP-CD8+ T cells ratio). The cytotoxicity of AP-CD8+ T cells toward LLC cells at 18 and 36 hrs after coculture was determined using LIVE/DEAD™ Fixable Violet Dead Cell Stain Kit and evaluated by BD LSRFortessa X-20 flow cytometer (BD Biosciences, San Jose, CA, USA). The specific death of GFP-LLC cells was identified by GFP+ LIVE/DEAD+ gating using BD FACSDiva software (BD Bioscience).

### Evaluation of the Effect of Peptide R10 on the Growth of Murine Lung Carcinoma Cells in Lung

The effect of designed peptide R10 was evaluated using an orthotopic LLC cell allograft growth in C57BL/6 mice (n=5-6). The mice were inoculated LLC cells (1.5×106 cells/200 μl PBS/mouse) via tail vein. After 5 days, JAWS-irrLLC (0.5×106 cells/200 μl PBS/mouse) prepared as same manner described above were intravenously injected into all mice via tail vein. After 2 days, mice were randomly separated into 3 groups and treated with (1) PBS as negative control, (2) peptide R10 (10 mg/kg/day, IP, every other day, 4 times), and (3) anti-PD-L1 antibody (αPD-L1; 10 mg/kg/day, IP, every two days, 3 times). At 23 days after LLC inoculation, all mice were sacrificed by cervical dislocation after exposure to saturated CO2 and blood was collected by cardiac puncture for flow cytometry analysis as described below. The lung and spleen were collected to examine their weights and fixed in 10% formalin for histological analysis.

### The Analysis of CD4 and CD8 T Cell Population and its Expression of Immune Check Point Molecules in Tumor-Bearing Mouse Blood by Flow Cytometry

After red blood cells (RBC) were removed using an RBC lysing buffer, the leukocytes were immune-stained using anti-CD4 (helper T cells), anti-FoxP3 (regulatory T cells), anti-CD8b (cytotoxic T cells), anti-IFNγ (activated cytotoxic T cells) antibodies for flow cytometric analysis of their population. The expression of immune checkpoint molecules was also evaluated using anti-CTLA4 and anti-PD-1 antibodies. Non-specific reaction of antibodies was evaluated by the isotype control. The percentage cell populations and mean fluorescence intensity (MFI) of immune check point molecules were analyzed using flow cytometer described above.

### Results

### Peptide R10 Treatment Increase Cytotoxicity of Antigen Primed CD8+ T Cells Toward LLC Cell

To evaluate effect of newly designed CTLA4 inhibitory peptide (R10) on cytotoxicity of antigen-specific cytotoxic CD8+ T cells, LLC cell antigen-primed CD8+ T cells (AP-CD8+ T cells) were generated by in vivo stimulation injecting with mouse immature dendritic cell line JAWSII cells cocultured with irradiated LLC cells (JAWS-irLLC). Resultant AP-CD8+ T cells was isolated from spleen, then the effect of designed peptide R10 on cytotoxicity of AP-CD8+ T cells toward LLC cells were evaluated by in vitro co-culture system and following flow cytometric analysis. As shown in FIG. 6, LLC-specific cell death at 18 hrs after co-culture was promoted in the presence of designed peptide R10 (10 μM) treated group (43.8±3.0% of cell death in total LLC, P<0.05 compared to PBS) compared to PBS treated group (12.7±0.3%). Although treatment with 1.0 μg/ml mouse anti-PD-L1 antibody (αPD-L1), which was used as positive control of immune check point inhibitor, showed highest efficacy on promoting AP-CD8+ T cells induced LLC cell death (66.6±1.5%, P<0.05 compared to all other groups), the effect of designed peptide R10 was same level with that of 0.5 μg/ml αPD-L1 (49.1±1.4%, P<0.05 compared to PBS). The effect of designed peptide R10 was sustained strongly at 36 hrs timepoint (68.2±12.3%, n.s.) after co-culture, while that of both 0.5 and 1.0 μg/ml αPD-L1 were decreased (27.2±8.7% and 27.5±12.9%, respectively). These results may suggest that designed peptide R10 effectively inhibit binding between CTLA4 on AP-CD8+ T cells and CD80/CD86 on LLC cancer cells, therefore cytotoxic effect of AP-CD8+ T cells was promoted.

### Designed Peptide R10 Treatment Attenuate the Growth of LLC Tumor in Mouse Lung

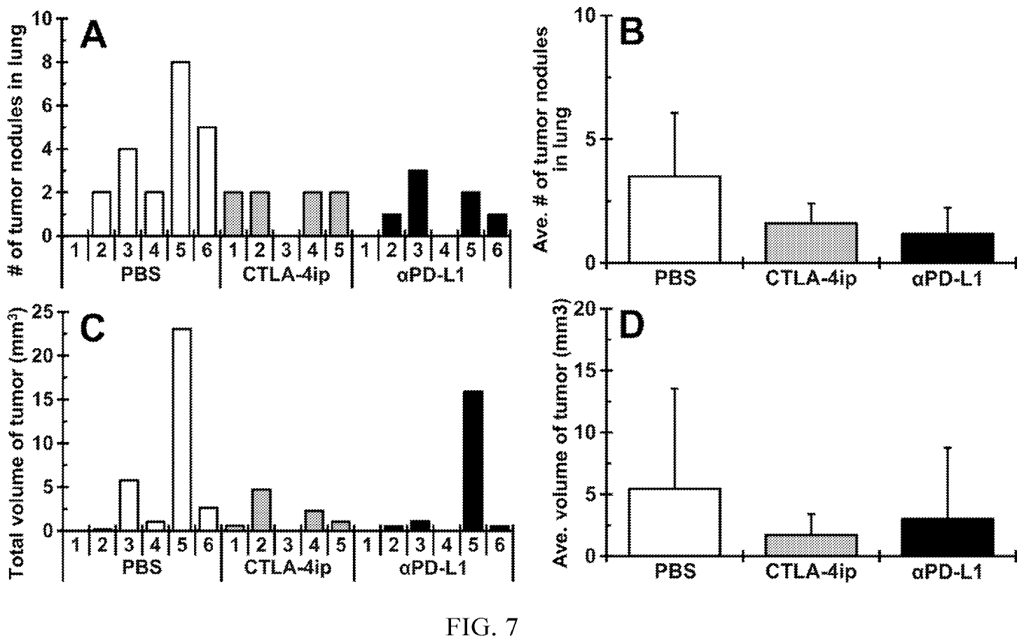

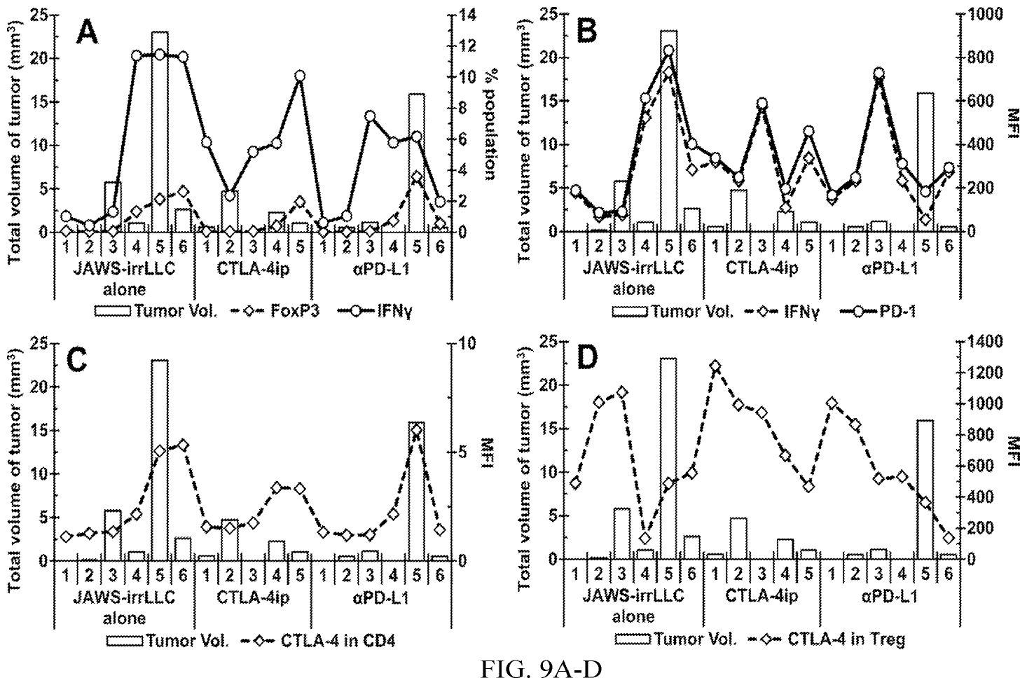

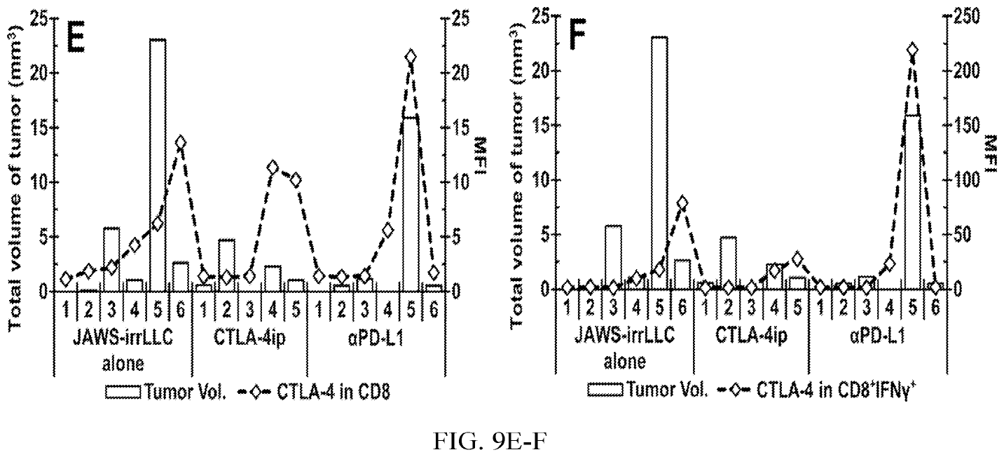

The effect of designed peptide R10 on the tumor growth in lung was evaluated using LLC mouse allograft model. To enhance antitumor immunity of host, mouse dendritic cell line, JAWSII cells were cocultured with irradiated LLC cells for 24 hrs (JAWS-irrLLC in FIG. 7) and injected into all mice at 5 days after intravenous inoculation of LLC cells. The treatment with CTLA4 inhibitory peptide (R10, 10 mg/kg/day) and anti-PD-L1 antibody (αPD-L1, 10mg/kg/day) was started from 2 days after JAWS-irLLC injection as described in Materials and Methods. In macroscopic observation, a smaller number of tumor nodules was confirmed in designed peptide R10 group (4 out of 5, Ave. 1.60±0.80) compared to that of in negative control, JAWS-irrLLC alone group (5 out of 6, Ave. 3.50±2.57), which was same level with that of positive control, αPD-L1 (4 out of 6, Ave. 1.17±1.07) (FIGS. 9A and B). These results suggest that novel designed peptide R10 promote anticancer immunity, therefore tumor growth was attenuated.

### Flow Cytometric Analysis of Immune Cell Population and Expression of Immune Check Point Molecules Leukocytes Collected From LLC Tumor-Bearing Mouse Blood

At the end of mouse study, peripheral blood was collected from each mouse. Leukocytes were isolated to evaluate the population change of CD4+ and CD8+ T cells, and expression status of immune checkpoint molecules by flow cytometry.

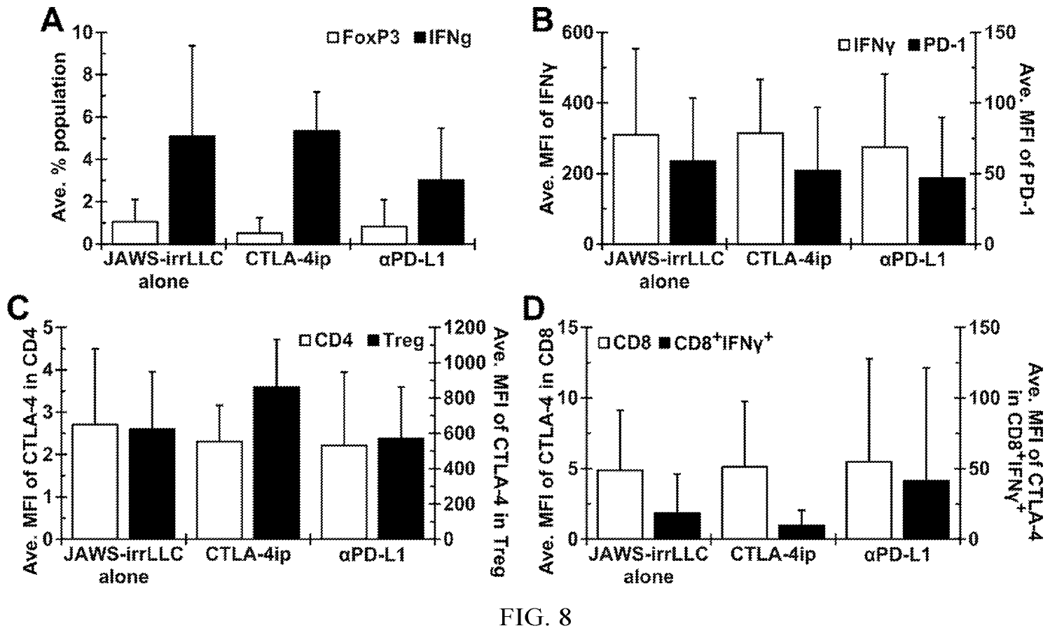

The treatment with designed peptide R10 decreased Treg population (0.50±0.74%) compared to that of non-treated JAWS-irrLLC alone (1.05±1.06%) and mouse anti-PD-L1 antibody (αPD-L1; 0.83±1.26%) (FIG. 8A). On the cytotoxic T lymphocyte (CTL) population, no difference was observed between JAWS-irrLLC alone group (5.10±4.26%) and designed peptide R10 group (5.33±1.85%), while the population was decreased in αPD-L1 group (3.01±2.45%). In the comparison of expression level (mean fluorescence intensity; MFI) of IFNγ and PD-1, clear differences were not observed between these groups (FIG. 8 B). The treatment with designed peptide R10 tended to increase CTLA4 expression in Treg (MFI: 863.02±269.34) compared to JAWS-irrLLC alone and αPD-L1 groups (MFI: 624.98±324.71 and 570.84±292.13, respectively), while the expression level of CTLA4 in Treg was not changed between these groups (FIG. 8 C). On the other hand, the treatment with designed peptide R10 tended to decrease CTLA4 expression in CD8+ IFNγ+ activated cytotoxic T cell (MFI: 9.71±10.79) compared to JAWS-irrLLC alone and αPD-L1 groups (MFI: 18.52±27.59 and 41.52±79.83, respectively) (FIG. 8 D). The treatments did not change that of in CD8+ cytotoxic T cell (FIG. 8 C). These results may suggest that immune suppressive effect of Treg toward CD8+ cytotoxic T cell was effectively inhibited by designed peptide R10 treatment, therefore tumor growth in the group was attenuated by strong anticancer immunity.

### Correlation Diagram Between Tumor Growth, Immune Cell Population and Expression of Immune Check Point Molecules

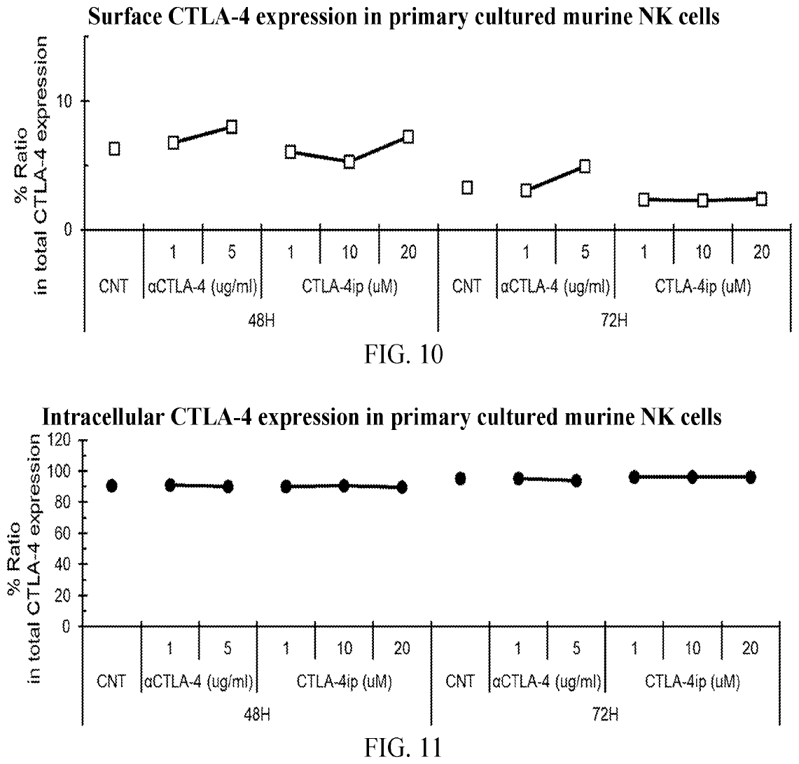

In the comparison of individual mouse, high population of Treg and CTL was observed in the mouse with high tumor growth (2.14 and 9.31% in JAWS-irrLLC alone #5 mouse, and 3.59 and 2.58% in αPD-L1 #5 mouse, respectively) (FIG. 9A). In the JAWS-irrLLC alone #5 mouse, high level of both IFNγ and PD-1 expression was observed in CD8+ cytotoxic T cell (FIG. 9 B). These results may suggest that tumor growth in the mouse was promoted by induction of exhaustion in CD8+ cytotoxic T cell. In contrast, both IFNγ and PD-1 expression was low in the αPD-L1 #5 mouse (FIG. 9 B). However, the expression of CTLA4 was increased in CD4+ cells (FIG. 9 C), CD8+ cytotoxic T cell (FIG. 9 E), and CD8+ IFNγ+ activated cytotoxic T cell (FIG. 9 F). Although the mouse was treated with αPD-L1, which is inhibitor of immune check point pathway: PD-1 and PD-L1 axis, the tumor growth may be promoted by inhibition of CD8+ cytotoxic T cell via immune check point pathway: CD80/CD86 and CTLA4 axis. As shown in FIGS. 10 C and 11 D, higher level of CTLA4 expression was observed in designed peptide R10 treated group compared to other two groups. However, tumor growth was inhibited as shown in FIG. 7. These results may suggest that immune suppressive effect of Treg toward CD8+ cytotoxic T cell was effectively inhibited by designed peptide R10 treatment, therefore tumor growth in the group was attenuated by strong anticancer immunity. These comparison analysis between tumor growth and leukocytes may support assumption described above on the mechanism of action of novel designed peptide R10 for tumor attenuation via anticancer immunity.

### Discussion

As we mentioned in the above section, only two peptides R7, SIITVLTPTGWVAAEFS (SEQ ID NO:31) and R10, have shown significant binding activity with CTLA-4 protein. The peptide R7 was too hydrophobic and could not be synthesized (and further would not be compatible with physiological conditions). Hence, we focused our study on the interaction of peptide R10 and CTLA-4.

### Interaction of Peptide R10 With CTLA4

Two microsecond long MD simulation yields configurations of the designed peptide (R10) at the binding surface of the CTLA4 where B-7 family protein binds. Computer modeling show three hydrogen bonds, one salt-bridge and one π-π stacking interaction between R10 peptide and CTLA4 protein (not shown). Modeling indicates a hydrogen bond between (A) the alcohol hydrogen atom of the sidechain of Thr53 of CTLA-4 and carbonyl oxygen of Ala13 of the peptide (B) hydrogen atom of the aliphatic sidechain of the Arg35 of CTLA4 and carbonyl oxygen atom of the Arg15 of the peptide and (C) oxygen atom of carboxyl group of Glu45 of CTLA4 and a hydrogen atom of the guanidinium group of Arg13 of peptide remain stable and stay bound for more than 1 microsecond. Most importantly, the aromatic ring of Tyr16 from R10 peptide occupies the space between the aromatic rings of Tyr101 and Try97 of conserved motif (MYPPPY (SEQ ID NO:18) loop) of CTLA4 protein and makes stable π-π stacking with Tyr 101 of CTLA4. A hydrophobic connection between hydrogen atoms from the aliphatic sidechain of the Pro8 of peptide and hydrogen atoms from the aliphatic sidechain of the Glu56 of the CTLA4 and stability of this interaction is depicted in the simulation.

### EXAMPLE 3

### Effect of CTLA-4ip on the CTLA-4 Expression in NK Cells

Natural Killer (NK) calls were isolated from C57BL/6 mouse spleen using MagniSort™ Mouse NK cell Enrichment Kit (ThermoFisher Scientific, Waltham, MA). The cells were seeded into 12-well plate and treated with 12.5 ng/ml recombinant mouse IL-2 for an induction of CTLA-4 protein expression; the expression of the CTLA-4 protein in cell surface of immune cells is very low under physiological conditions. After 24 hours, the cells were treated with 1, 10, and 20 μM CTLA-4ip. Anti-CTLA-4 antibody (aCTLA-4, 1 and 5 μg/ml) was used as a positive control treatment. PBS was used as a negative control. At 48 and 72 hours after various agent treatments, CTLA-4 expression was evaluated by Flow cytometry. The surface CTLA-4 on the cells were first stained with BV421-conjugated anti-CTLA-4 antibodies (UC10-4B9 clone). The cells were fixed with Fixation Buffer (BioLegend, San Diego, CA), permeabilizated by Intracellular Staining Permeabilization Wash Buffer (BioLegend), and stained with APC-conjugated anti-CTLA-4 antibodies (UC10-4B9 clone).

The results of surface and intracellular expression of CTLA-4 expression are shown in FIGS. 10 and 11. The results show that treatment with anti-CTLA-4 antibody increases CTLA-4 expression in cell surface approximately 49% within 72 hour incubation in cell culture in NK cells from the spleen. The study with CD8+ T cells from the spleen are under the investigation. However, treatment with CTLA-4ip peptide caused a negligible change (4.3%). These results remained consistent in additional experiments out to day 5 and day 7, with surface and intracellular expression of CLTA-4 remaining unchanged with CTLA-4ip. This suggests that the treatment with anti-CTLA-4 antibody stimulates a compensatory mechanisms of immune checkpoint system and weaken the anti-tumor immunity, but CTLA-4ip does not induce this compensatory mechanism. Most of cancer cells including lung cancer cells also express CTLA-4 on their cell surface, but their changes by anti-CTLA-4 antibody and CTLA-4ip are small (less than 6%). If the dosages of the anti-CTLA-4 antibody (aCTLA-4, 1 and 5 μg/ml) are comparable to those for human use, our finding strongly supports a better usability of our CTLA-4 inhibitory peptide over anti-CTLA-4 antibody (clinically available therapeutics).

### EXAMPLE 4

### Cytotoxicity Assay With H1N1 NK Cells and H1N1 Infected CT26 Cells

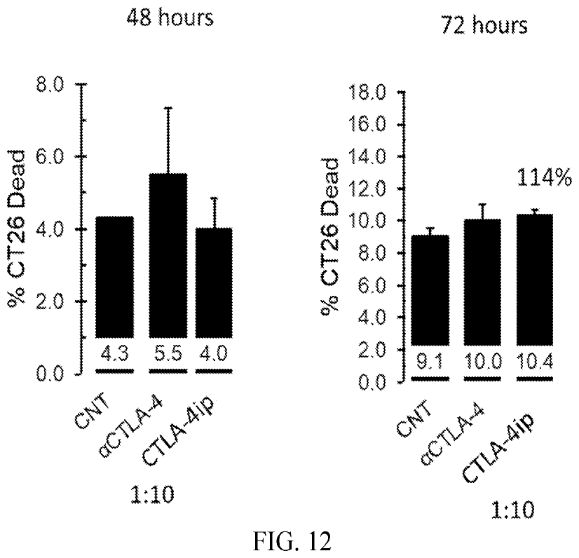

In this Example, CT26 cells were seeded in a 12-well plate 2 days before the assay. Cells were infected with 1/100 diluted H1N1 influenza virus. NK cells were cocultured with the H1N1-CT26 cells in a 1:10 ratio (n=2), and cells were then treated with either 5 μg/mL anti-CLTA-4 antibody (αCTLA-4), 10 μM CTLA-4ip, or 12.5 ng/mL rmIL-2 at Day 0. On Days 2 and 3, the cytotoxicity of the NK cells for the H1N1-infected CT26 cells was evaluated by flow cytometry. The results are shown in FIG. 12. The results show inhibitory activity better than the positive control at 72 hours and increased cytotoxicity of NK cells and increase clearing of influenza infected cells.

### EXAMPLE 5

### Cellular Uptake and Enzymatic Degradation—Biological Stability

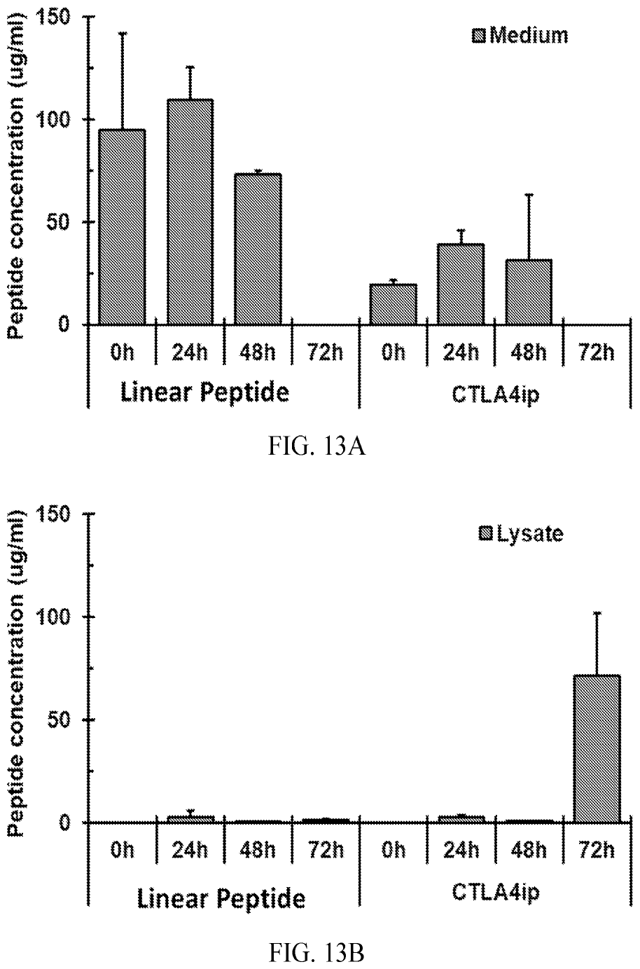

In this Example, CTLA-4ip was incubated with cells along with a control linear peptide of the same length, and peptide concentration was measured over time LC-MS. The results are shown in FIGS. 13A and 13B. As can be seen over time, the peptide concentration in the cell culture media is depleted over time. However, in the case of the cyclic peptide, it remains detectable in its cyclic form at high level in the cells, indicating both that it is taken up by the cells and further is resistant to enzymatic degradation over time.

1. An immune checkpoint inhibitor oligopeptide comprising the sequence:
R10: EIDTVLTPTGWVAKRYS (SEQ ID NO:1).

2. The immune checkpoint inhibitor oligopeptide of claim 1, wherein said oligopeptide is a cyclic peptide.

3. The immune checkpoint inhibitor oligopeptide of claim 2, wherein said cyclic peptide comprises the N-terminal head of said sequence connected to the C-terminal tail of said sequence via a peptide bond.

4. The immune checkpoint inhibitor oligopeptide of claim 1, further comprising a tumor targeting moiety conjugated thereto.

5. The immune checkpoint inhibitor oligopeptide of claim 1 in a unit dosage form.

6. A therapeutic composition comprising a plurality of immune checkpoint inhibitor oligopeptides according to claim 1, said oligopeptides dispersed in a pharmaceutically-acceptable carrier.

7. The therapeutic composition of claim 6, comprising a therapeutically effective amount of said oligopeptide.

8. The therapeutic composition of claim 6, wherein said carrier is selected from the group consisting of normal (n.) saline (˜0.9% NaCl), phosphate buffered saline (PBS), sterile water/distilled autoclaved water (DAW), various oil-in-water or water-in-oil emulsions, as well as dimethyl sulfoxide (DMSO), and other acceptable vehicles.

9. The therapeutic composition of claim 6, further comprising one or more additional ingredients selected from the group consisting of other checkpoint inhibitors, immunotherapies, chemotherapies, preservatives, buffering agents, salts, and mixtures thereof.

10. The therapeutic composition of claim 9, wherein said other checkpoint inhibitors are inhibitors targeting PD-1.

11. The therapeutic composition of claim 9, wherein said immunotherapies comprise monoclonal antibodies.

12. A method of treating cancer, said method comprising administering a plurality of immune checkpoint inhibitor oligopeptides according to claim 1 to a subject in need thereof.

13. The method of claim 12, wherein said plurality of immune checkpoint inhibitor oligopeptides enhance an immune response against cancer cells in said subject.

14. The method of claim 12, wherein said plurality of immune checkpoint inhibitor oligopeptides stimulate an immune response against cancer cells in said subject.

15. The method of claim 12, wherein a therapeutically-effective amount of said plurality of immune checkpoint inhibitor oligopeptides is administered to said subject.

16. The method of claim 12, wherein a composition comprising a therapeutically-effective amount of said plurality of immune checkpoint inhibitor oligopeptides is administered to said subject.

17. The method of claim 12, wherein said plurality of immune checkpoint inhibitor oligopeptides are administered intramuscularly, subcutaneously, intradermally, intravenously, mucosally, topically, or intratumorally.

18. The method of claim 12, wherein said plurality of immune checkpoint inhibitor oligopeptides are coadministered with other immunotherapies and/or chemotherapies.

19. The method of claim 12, wherein said plurality of immune checkpoint inhibitor oligopeptides are administered as a primary cancer treatment.

20. The method of claim 12, wherein said plurality of immune checkpoint inhibitor oligopeptides are administered as an adjunctive treatment.

21. The method of claim 20, where said plurality of immune checkpoint inhibitor oligopeptides are administered as an adjunctive treatment to a primary treatment comprising chemotherapy, radiation therapy, hormone therapy, and/or immunotherapy.

22. The method of claim 12, wherein said plurality of immune checkpoint inhibitor oligopeptides stimulate an increased immune response in said subject for at least 24 hours after administration.

23. The method of claim 12, wherein said subject exhibits a reduction in cancer cell numbers, a reduction in tumor nodule numbers, a reduction in volume of cancer cells, a reduction in tumor volume, and/or a reduction in rate of growth of said cancer cells or tumor after said administration.

24. A kit comprising a plurality of immune checkpoint inhibitor oligopeptides according to claim 1 in a unit dosage form in a container, and instructions for administering said plurality of immune checkpoint inhibitor oligopeptides to a subject in need thereof.

25. The kit of claim 24, wherein said plurality of immune checkpoint inhibitor oligopeptides are lyophilized powder.

26. The kit of claim 25, further comprising instructions for reconstituting said lyophilized powder in a pharmaceutically-acceptable carrier before administering to a subject in need thereof.

27. The kit of claim 24, wherein said unit dosage form comprises said plurality of immune checkpoint inhibitor oligopeptides dispersed in a pharmaceutically-acceptable carrier in said container.

28. A diagnostic kit to detect the presence or measure quantity of Cytotoxic T-Lymphocytes or Natural Killer Cells in a sample, said kit comprising a plurality of immune checkpoint inhibitor oligopeptides according to claim 1, said oligopeptides have at least one detectable label; and instructions for use thereof.

29. (canceled)

30. A medicament for use in treating cancer, inhibiting growth of cancer cells, or enhancing cancer treatment, comprising an immune checkpoint inhibitor oligopeptide according to claim 1.


SEQUENCE LISTING (35 sequences)

SEQ ID NO: 1 - protein, 17 aa, Artificial Sequence
  Synthetic inhibitor peptide
EIDTVLTPTGWVAKRYS

SEQ ID NO: 2 - protein, 17 aa, Artificial Sequence
  Synthetic inhibitor peptide
IDTVLTPTGWVAKRYSE

SEQ ID NO: 3 - protein, 17 aa, Artificial Sequence
  Synthetic inhibitor peptide
DTVLTPTGWVAKRYSEI

SEQ ID NO: 4 - protein, 17 aa, Artificial Sequence
  Synthetic inhibitor peptide
TVLTPTGWVAKRYSEID

SEQ ID NO: 5 - protein, 17 aa, Artificial Sequence
  Synthetic inhibitor peptide
VLTPTGWVAKRYSEIDT

SEQ ID NO: 6 - protein, 17 aa, Artificial Sequence
  Synthetic inhibitor peptide
LTPTGWVAKRYSEIDTV

SEQ ID NO: 7 - protein, 17 aa, Artificial Sequence
  Synthetic inhibitor peptide
TPTGWVAKRYSEIDTVL

SEQ ID NO: 8 - protein, 17 aa, Artificial Sequence
  Synthetic inhibitor peptide
PTGWVAKRYSEIDTVLT

SEQ ID NO: 9 - protein, 17 aa, Artificial Sequence
  Synthetic inhibitor peptide
TGWVAKRYSEIDTVLTP

SEQ ID NO: 10 - protein, 17 aa, Artificial Sequence
  Synthetic inhibitor peptide
GWVAKRYSEIDTVLTPT

SEQ ID NO: 11 - protein, 17 aa, Artificial Sequence
  Synthetic inhibitor peptide
WVAKRYSEIDTVLTPTG

SEQ ID NO: 12 - protein, 17 aa, Artificial Sequence
  Synthetic inhibitor peptide
VAKRYSEIDTVLTPTGW

SEQ ID NO: 13 - protein, 17 aa, Artificial Sequence
  Synthetic inhibitor peptide
AKRYSEIDTVLTPTGWV

SEQ ID NO: 14 - protein, 17 aa, Artificial Sequence
  Synthetic inhibitor peptide
KRYSEIDTVLTPTGWVA

SEQ ID NO: 15 - protein, 17 aa, Artificial Sequence
  Synthetic inhibitor peptide
RYSEIDTVLTPTGWVAK

SEQ ID NO: 16 - protein, 17 aa, Artificial Sequence
  Synthetic inhibitor peptide
YSEIDTVLTPTGWVAKR

SEQ ID NO: 17 - protein, 17 aa, Artificial Sequence
  Synthetic inhibitor peptide
SEIDTVLTPTGWVAKRY

SEQ ID NO: 18 - protein, 6 aa, Homo sapiens
MYPPPY

SEQ ID NO: 19 - protein, 17 aa, Homo sapiens
CIIHHKKPTGMIRIHQM

SEQ ID NO: 20 - protein, 17 aa, Artificial Sequence
  Synthetic peptide
ECRYEPRPEGNILVSYS

SEQ ID NO: 21 - protein, 17 aa, Artificial Sequence
  Synthetic peptide
SIVTKLTPTGWVAASYS

SEQ ID NO: 22 - protein, 17 aa, Artificial Sequence
  Synthetic peptide
KVEFKRTPSGTITVSME

SEQ ID NO: 23 - protein, 17 aa, Artificial Sequence
  Synthetic peptide
KVVYEPKPEGNIVVEYE

SEQ ID NO: 24 - protein, 17 aa, Artificial Sequence
  Synthetic peptide
SAKFEPRPEGNIVVSYG

SEQ ID NO: 25 - protein, 17 aa, Artificial Sequence
  Synthetic peptide
EARYQPRPDGNVLVSYG

SEQ ID NO: 26 - protein, 17 aa, Artificial Sequence
  Synthetic peptide
SAKWNPKPEGAELIEEG

SEQ ID NO: 27 - protein, 17 aa, Artificial Sequence
  Synthetic peptide
SAEFIPTPDGNLLKSSG

SEQ ID NO: 28 - protein, 17 aa, Artificial Sequence
  Synthetic peptide
SIVVVLTPTGWVAASYS

SEQ ID NO: 29 - protein, 17 aa, Artificial Sequence
  Synthetic peptide
EIITKLTPTGWVAASYS

SEQ ID NO: 30 - protein, 17 aa, Artificial Sequence
  Synthetic peptide
SIEMELTPTGWVNKSSS

SEQ ID NO: 31 - protein, 17 aa, Artificial Sequence
  Synthetic peptide
SIITVLTPTGWVAAEFS

SEQ ID NO: 32 - protein, 17 aa, Artificial Sequence
  Synthetic peptide
DIITILTPTGYVAAAYS

SEQ ID NO: 33 - protein, 17 aa, Artificial Sequence
  Synthetic peptide
SIITVLTPTGWVAAYYS

SEQ ID NO: 34 - protein, 17 aa, Artificial Sequence
  Synthetic peptide
SIQCVLTPTGWVAARYS

SEQ ID NO: 35 - protein, 17 aa, Artificial Sequence
  Synthetic peptide
SIRMELTPTGWVAAEYE